(19) United States
(12) Patent Application Publication
(10) Pub. No.: US 2014/0093928 A1
(43) Pub. Date: Apr. 3, 2014

(54) Methods and Systems for Biologically Producing Methanol

(71) Applicant: Kartik Chandran, New York, NY (US)

(72) Inventor: Kartik Chandran, New York, NY (US)

(21) Appl. No.: 13/991,921

(22) PCT Filed: Dec. 8, 2011
(86) PCT No.: PCT/US11/63911

§ 371 Date: Dec. 17, 2013

Related U.S. Application Data
(60) Provisional application No. 61/420,848, filed on Dec. 8, 2010.
(60) Provisional application No. 61/546,278, filed on Oct. 12, 2011.

(51) Int. Cl. C12P 7/04

(52) U.S. Cl. CPC C12P 7/04
USPC 435/157; 435/289.1

(57) ABSTRACT

A method for biologically producing methanol is disclosed. In some embodiments, the method includes the following: providing a biomass including ammonia oxidizing bacteria having ammonia monooxygenase enzymes and hydroxylamine oxidoreductase enzymes; feeding ammonia to the ammonia oxidizing bacteria; feeding a non-substrate-organic compound including methane to the ammonia oxidizing bacteria; feeding oxygen and reducing equivalents (in the form of hydroxylamine) to the ammonia oxidizing bacteria; oxidizing the ammonia using the ammonia monooxygenase enzymes in the ammonia oxidizing bacteria to generate hydroxylamine and oxidizing the hydroxylamine using the hydroxylamine oxidoreductase enzymes to form nitrite; and partially oxidizing the methane in the compound using the ammonia monooxygenase enzymes in the ammonia oxidizing bacteria to generate methanol.

### CROSS REFERENCE TO RELATED APPLICATION(S)

This application claims the benefit of U.S. Provisional Application No. 61/420,848, filed Dec. 8, 2011 and U.S. Provisional Application No. 61/546,278, filed Oct. 12, 2011, each of which is incorporated by reference as if disclosed herein in its entirety.

### BACKGROUND

The primary limitations in the widespread use of methane gas to produce energy include the cost of its chemical purification and challenges of handling a gaseous phase stream. One possible mechanism to overcome these limitations is to convert methane to a more readily usable liquid-fuel such as methanol. Currently in the United States, methanol is produced through chemically catalyzed conversion of methane gas. However, the chemical technologies to convert methane to methanol are not the most economical. Additionally, sources of methane such anaerobic digester gas still need purification prior to chemical conversion to methanol.

The United States is investing significant resources to become a leader in bio-based fuels and energy. While ethanol has been of primary focus, it should be noted that other biofuels such as methanol can be also equally if not even more attractive. Methanol is widely used as an additive in gasoline blends, as an electron donor in fuel cells, as a trans-esterfication agent to convert long-chain fatty acids and lipids to biodiesel and as a precursor to synthesize dimethyl ether (DME). In addition, methanol is still one of the most widely used chemicals for enhancing denitrification in wastewater treatment. Most methanol in the United States is produced by chemical oxidation of methane. The chemical catalysis pathway is expensive, energy intensive, and redundant; involving initial oxidation of methane to carbon dioxide and hydrogen and then reduction of carbon dioxide back to methanol.

### SUMMARY

Methane is typically the primary energy substrate for methane oxidizing bacteria. However, methane oxidizing bacteria oxidize methane completely to carbon dioxide, which again is gaseous and cannot be directly used as a biofuel. On the other hand, ammonia oxidizing bacteria, which “co-metabolize” methane can only partially oxidize it to methanol (and trace amounts of formaldehyde). Based on known genomes of ammonia oxidizing bacteria, they in fact completely lack the capacity to produce carbon dioxide from methane. Additionally, carbon dioxide, which often limits direct combustion of digester gas, is used by ammonia oxidizing bacteria as a growth substrate, i.e., by fixing it to produce more cells. As shown schematically in FIG. 1, aspects of the disclosed subject matter include methods and systems for employing mono-oxygenic pathways in ammonia oxidizing bacteria to biologically oxidize methane to methanol, which can be directly used as a biofuel, and not completely to carbon dioxide.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1 is a schematic diagram of systems and methods including energy and co-metabolism in ammonia oxidizing bacteria using ammonia and methane to generate methanol according to some embodiments of the disclosed subject matter; and

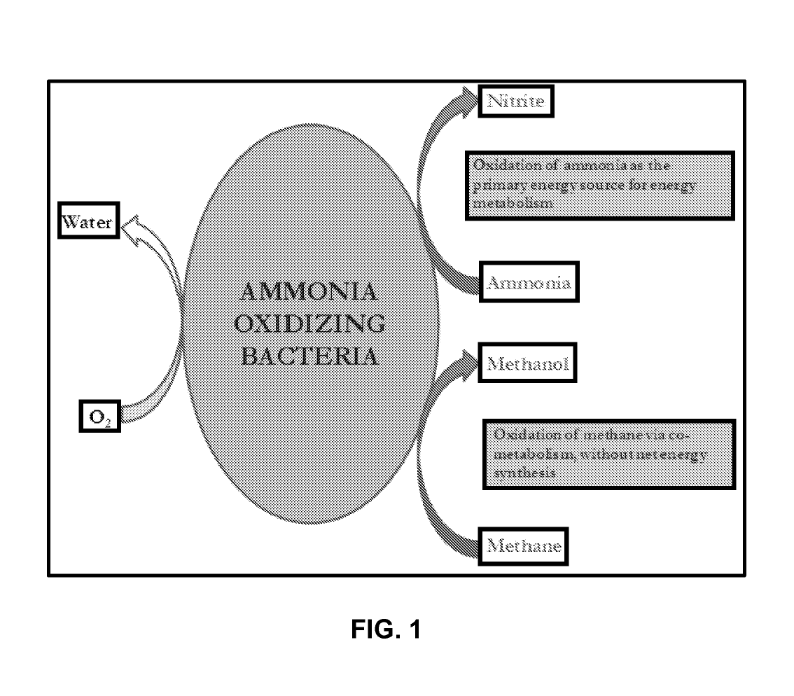

FIG. 2 is a schematic diagram of systems according to some embodiments of the disclosed subject matter;

FIG. 3 is a schematic diagram of metabolic pathways of the ammonia oxidizing bacteria N. europaea; and

FIG. 4 is a chart of a method according to some embodiments of the disclosed subject matter.

### DETAILED DESCRIPTION

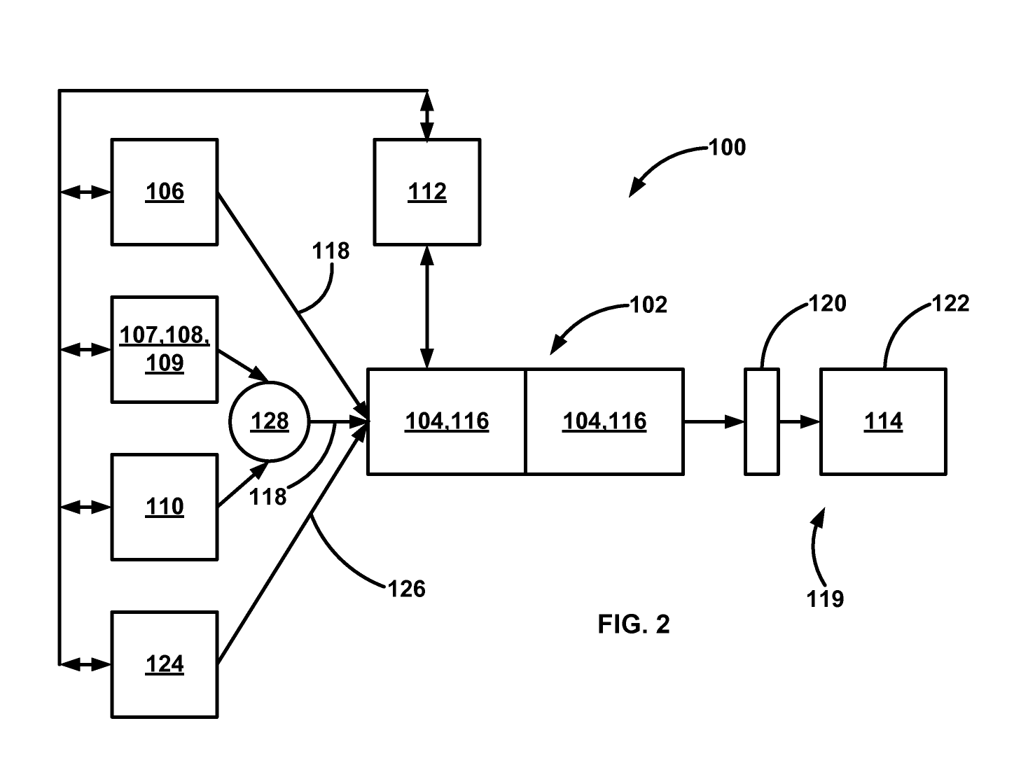

Referring now to FIG. 2, aspects of the disclosed subject matter include a system 100 for biologically producing methanol. System 100 includes a bio reactor 102 for reacting a biomass 104 with ammonia 106, a compound 107 including methane 108, hydroxylamine 109 and oxygen 110, and a control sub-system 112 for controlling the reactions within the bio reactor to optimize the generation of methanol 114.

Reactor 102 contains biomass 104. A portion of biomass 104 includes ammonia oxidizing bacteria 116 in wild type and genetically modified forms, which have ammonia monooxygenase enzymes and hydroxylamine oxidoreductase enzymes. In some embodiments, bacteria 116 include at least one or several of N. europaea, N. eutropha, N. oligotropha, Nitrosospira multiformis as well as nitrite oxidizing bacteria, Nitrobacter winogradskyi and Nitrospira spp. as needed.

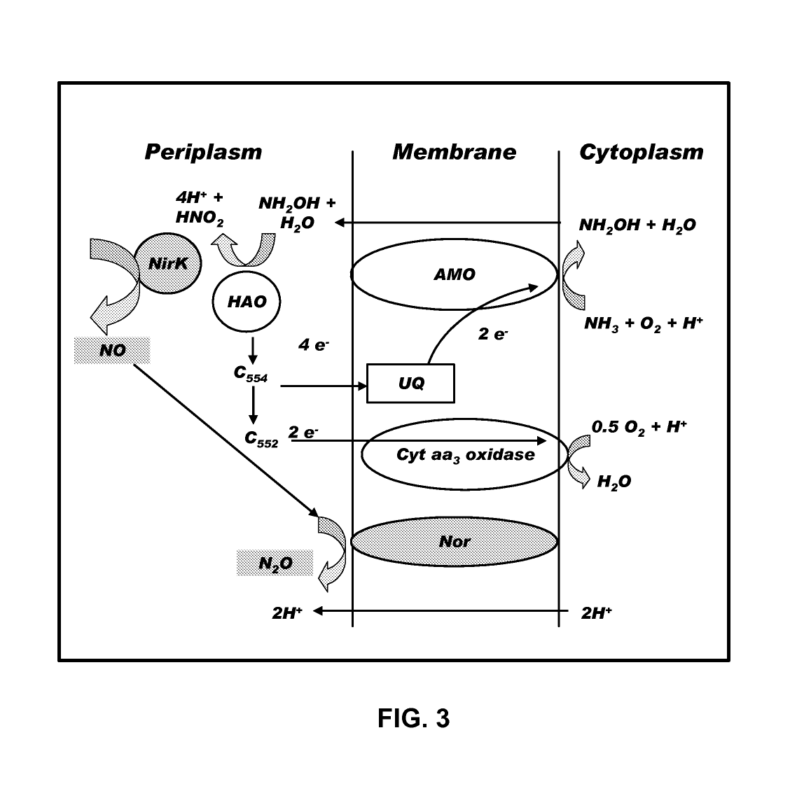

Referring now to FIG. 3, the metabolism of ammonia oxidizing bacteria is shown. The oxidation of ammonia-nitrogen (NH₃—N) to nitrite-nitrogen (NO2—N) serves as the primary source of energy for ammonia oxidizing bacteria. NH₃—N is oxidized to hydroxylamine (NH₂OH) by membrane-bound ammonia monooxygenase (AMO) and NH₂OH is oxidized to NO₂—N by hydroxylamine oxidoreductase (HAO). The dominant mode of energy generation by ammonia oxidizing bacteria is via aerobic metabolic pathways (un-shaded enzymes). However, under oxygen limiting conditions, ammonia oxidizing bacteria can utilize alternate electron acceptors such as NO₂— and produce nitrous oxide (N₂O) and nitric oxide (NO), which are shown in grey.

Fortuitous oxidation of organic compounds is also achieved by ammonia oxidizing bacteria: In addition to oxidizing ammonia, ammonia oxidizing bacteria can also oxidize other non-substrate-organic compounds, including methane and various other hydrocarbons. This is because of the poor substrate specificity of the enzyme ammonia monooxygenase (AMO). The ability of AMO to oxidize methane is thought to be because of its close relation to methane monooxygenase (MMO) from an evolutionary perspective. MMO is used by methane oxidizing bacteria for energy production.

In methods and systems according to the disclosed subject matter, ammonia oxidizing bacteria are preferred over methane oxidizing bacteria to oxidize methane to methanol. Since methane is the primary energy substrate for methane oxidizing bacteria, they oxidize methane completely to carbon dioxide, which cannot be used as a fuel. On the other hand, methane is not the primary energy substrate for ammonia oxidizing bacteria. Ammonia oxidizing bacteria lack the capacity to produce carbon dioxide from methane and instead only oxidize methane partially to methanol, which can be used as a fuel.

Referring again to FIG. 2, reactor 102 is typically configured so that oxidation of ammonia 106 is isolated from oxidation of methane 108 within the reactor, e.g., separate chambers, staged oxidation, etc.

Sources of ammonia 106, non-substrate-organic compound 107 including methane 108, and oxygen 110 are fluidly connected to and supplied to reactor 102 via a conduit 118, e.g., pipe, etc. In some embodiments, compound 107 includes hydroxylamine 109 and carbon dioxide.

Control sub-system 112 is connected with reactor 102 and the sources of ammonia 106, compound 107, and oxygen 110. Control sub-system 112 is configured to provide/direct particular amounts of ammonia 106, compound 107, and oxygen 110 such that the ammonia is oxidized using the ammonia monooxygenase enzymes in ammonia oxidizing bacteria 116 to generate hydroxylamine. The hydroxylamine is oxidized using the hydroxylamine oxidoreductase enzymes in bacteria 116 to form nitrite. Finally, methane 108 in compound 107 is partially oxidized using the ammonia monooxygenase enzymes in ammonia oxidizing bacteria 116 to generate methanol 114.

In some embodiments, system 100 includes a methanol collection sub-system 119 having one or more filters 120 for separating methanol 114 from other constituents in reactor 102 and a compartment 122 for storing the methanol.

In some embodiments, system 100 includes a source of reductant 124 such as hydroxylamine in addition to that contained in ammonia 106. Reductant 124 is fluidly connected with reactor 102 via a conduit 126, e.g., piping, etc.

In some embodiments, system 100 includes a sparging sub-system 128 for supplying compound 107 and oxygen 110 to reactor 102 by continuously sparging the compound and the oxygen into biomass 104.

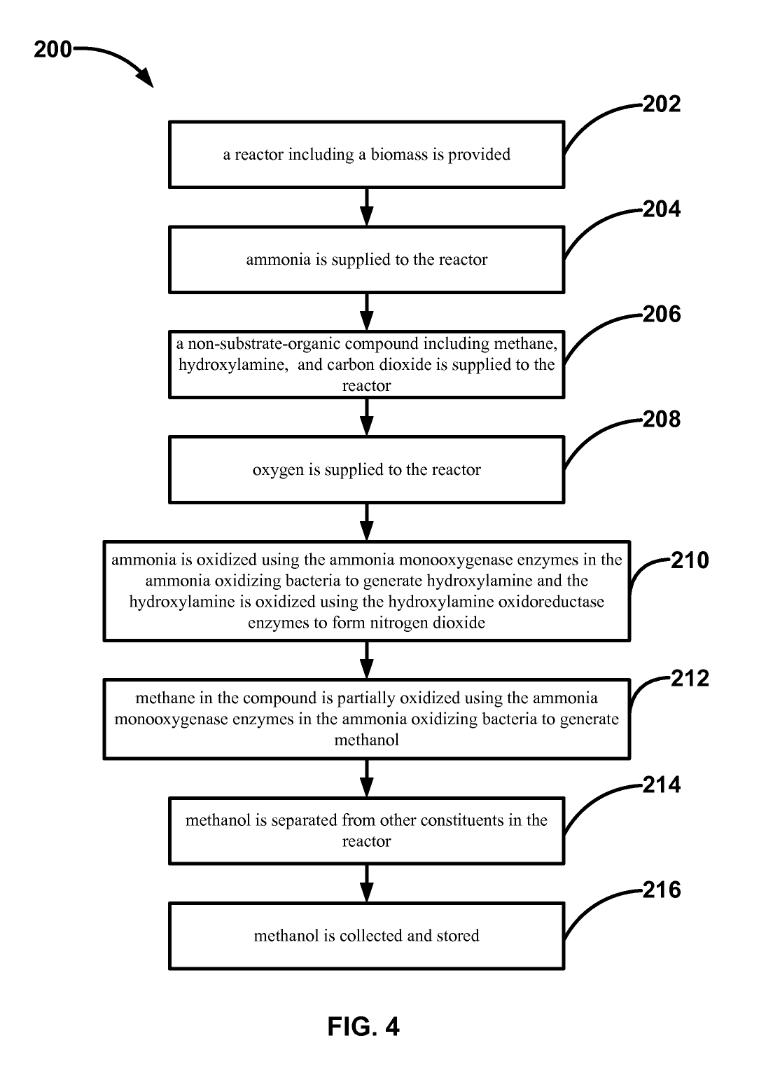

Referring now to FIG. 4, some embodiments of the disclosed subject matter include a method 200 for biologically producing methanol. At 202, a reactor including a biomass is provided. A portion of the biomass includes ammonia oxidizing bacteria having ammonia monooxygenase enzymes and hydroxylamine oxidoreductase enzymes. In some embodiments, the bacteria include at least one or several of N. europaea, N. eutropha, N. oligotropha Nitrosospira multiformis, Nitrobacter winogradskyi and Nitrospira spp. Then, at 204, ammonia is supplied to the reactor. At 206, a non-substrate-organic compound including methane and carbon dioxide is supplied to the reactor. At 208, oxygen is supplied to the reactor. Typically, amounts of the non-substrate-organic compound including methane and the oxygen supplied to the reactor are sufficient to saturate the biomass. In some embodiments, the compound and the oxygen are continuously sparged either together or in isolation, with the addition of hydroxylamine into the biomass. At 210, the ammonia is oxidized using the ammonia monooxygenase enzymes in the ammonia oxidizing bacteria to generate hydroxylamine and the hydroxylamine is oxidized using the hydroxylamine oxidoreductase enzymes to form nitrite. In some embodiments, hydroxylamine is also dosed externally into the biomass. At 212, the methane in the compound is partially oxidized using the ammonia monooxygenase enzymes in the ammonia oxidizing bacteria to generate methanol. In some embodiments, oxidation of the ammonia is isolated from oxidation of the methane within the reactor. At 214, the methanol is separated from other constituents in the reactor. At 216, the methanol is collected and stored. In some embodiments, method 200 includes supplying reductant such as hydroxylamine in addition to that contained in the ammonia to the reactor and/or producing reductant in the reactor in addition to that contained in the ammonia by keeping nitrogen oxidizing bacteria in the reactor in solution. In some embodiments

Methods and systems according to the disclosed subject matter enable the harnessing of digester gas into an operationally useful biofuel or external COD source in the form of methanol. Essentially by ‘integrating’ the microbial nitrogen and carbon cycles it is possible to simultaneously address nitrogen pollution (by conversion to nitrite using ammonia oxidizing bacteria) and channeling the methanol that ammonia oxidizing bacteria produce either to biofuel or using it to enhance denitrification of the nitrite thus produced. The production of methanol using autotrophic pathways is significant, since it also achieves biological carbon dioxide fixation. Consequently, the overall greenhouse footprints of wastewater treatment plants are reduced, i.e., by lowering both methane and carbon dioxide release as well as recovering methanol.

Methods and systems according to the disclosed subject matter can be used the conversion of any gaseous stream that contains methane with or without carbon dioxide and liquid streams containing ammonia to the biofuel, methanol, as well as other precursors of longer chain organic compounds (including alcohols and acids). Methods and systems according to the disclosed subject matter can be used to transform anaerobic digesters at wastewater treatment plants, landfills, peatbogs, and marshes, where production of methane is common, into biofuels and sources of biofuels, and thus converting them into biorefineries. Specifically, the methanol and longer chain organic acids and alcohols can be used for the following: 1. external carbon source for denitrification (market size ˜$50 million per year in the US alone); 2. Gasoline additives (Being done in large scale in China); 3. Pre-cursor for biodiesel production (biodiesel is one of the most rapidly growing fuels today); 4. Production of dimethylether (another biofuel); and 5. Precursor chemicals for chemical fuel cells. Methods and systems according to the disclosed subject matter allow for “biological” conversion of methane, a greenhouse gas, into a range of green fuels including methanol, DME and longer chain organic compounds. By using waste sources of methane, the reliance on natural gas is minimized. Waste processing facilities are converted to biorefineries.

Methods and systems according to the disclosed subject matter are completely biological, whereas the closest technologies are chemical. Methods and systems according to the disclosed subject matter also do not require clean methane and can be used to process digester gas, landfill gas, gas from peatbogs and marshes, without any cleaning or dehumidification. Methods and systems according to the disclosed subject matter are more cost effective than competing processes and require significantly less energy and resources for gas clean up. Transportation costs for the biofuels produced are also reduced for some industries such as wastewater treatment, since production is onsite at wastewater treatment plants and landfills.

Although the disclosed subject matter has been described and illustrated with respect to embodiments thereof, it should be understood by those skilled in the art that features of the disclosed embodiments can be combined, rearranged, etc., to produce additional embodiments within the scope of the invention, and that various other changes, omissions, and additions may be made therein and thereto, without parting from the spirit and scope of the present invention.

What is claimed is:

1. A method for biologically producing methanol, said method comprising:
providing a biomass including ammonia oxidizing bacteria having ammonia monooxygenase enzymes and hydroxylamine oxidoreductase enzymes;
feeding ammonia to said ammonia oxidizing bacteria;
feeding a non-substrate-organic compound including methane to said ammonia oxidizing bacteria;
feeding oxygen to said ammonia oxidizing bacteria;
oxidizing said ammonia using said ammonia monooxygenase enzymes in said ammonia oxidizing bacteria to generate hydroxylamine and oxidizing said hydroxylamine using said hydroxylamine oxidoreductase enzymes to form nitrite; and
partially oxidizing said methane in said compound using said ammonia monooxygenase enzymes in said ammonia oxidizing bacteria to generate methanol.

2. The method according to claim 1, further comprising isolating oxidizing of said ammonia from oxidizing of said methane.

3. The method according to claim 1, wherein said compound includes carbon dioxide.

4. The method of claim 1, further comprising:
separating said methanol; and
collecting said methanol.

5. The method of claim 1, wherein said bacteria include at least one of N. europaea, N. eutropha, Nitrosospira multiformis, N. oligotropha, Nitrobaster winogradskyi and Nitrospira spp.

6. The method of claim 1, further comprising:
supplying reductant in addition to that contained in said ammonia to said reactor.

7. The method of claim 1, further comprising:
producing reductant in said reactor in addition to that contained in said ammonia by keeping nitrogen oxidizing bacteria in said reactor in solution.

8. The method of claim 1, wherein amounts of said compound and said oxygen supplied to said reactor are sufficient to saturate said biomass.

9. The method according to claim 1, further comprising continuously sparging said compound and said oxygen into said biomass.

10. A system for biologically producing methanol, said system comprising:
a reactor including a biomass, a portion of which includes ammonia oxidizing bacteria having ammonia monooxygenase enzymes and hydroxylamine oxidoreductase enzymes;
a source of ammonia fluidly connected to said reactor;
a source of a non-substrate-organic compound including methane fluidly connected to said reactor;
a source of oxygen fluidly connected to said reactor; and
a control sub-system connected with said reactor, said source of ammonia, said source of a non-substrate-organic compound, and said source of oxygen, wherein said control sub-system is configured to provide an amount of said ammonia, an amount of said compound, and an amount of said oxygen such that said ammonia is oxidized using said ammonia monooxygenase enzymes in said ammonia oxidizing bacteria to generate hydroxylamine, said hydroxylamine is oxidized using said hydroxylamine oxidoreductase enzymes to form nitrite, and said methane in said compound is partially oxidized using said ammonia monooxygenase enzymes in said ammonia oxidizing bacteria to generate methanol.

11. The system of claim 10, wherein said reactor is configured so that oxidation of said ammonia is isolated from oxidation of said methane within said reactor.

12. The system of claim 10, wherein said compound includes carbon dioxide.

13. The system of claim 10, including a methanol collection sub-system comprising:
one or more filters for separating said methanol from other constituents in said reactor; and
a compartment for storing said methanol.

14. The system of claim 10, wherein said bacteria include at least one of N. europaea, N. eutropha, Nitrosospira multiformis, N. oligotropha, Nitrobaster winogradskyi and Nitrospira spp.

15. The system of claim 10, further comprising:
a source of reductant such as hydroxylamine in addition to that contained in said ammonia fluidly connected with said reactor.

16. The system of claim 10, further comprising:
a sparging sub-system for supplying said compound and said oxygen to said reactor by continuously sparging said compound and said oxygen into said biomass.

17. A method for biologically producing methanol, said method comprising:
providing a reactor including a biomass, a portion of which includes ammonia oxidizing bacteria having ammonia monooxygenase enzymes and hydroxylamine oxidoreductase enzymes;
supplying ammonia to said reactor;
supplying a non-substrate-organic compound including methane and carbon dioxide to said reactor;
supplying oxygen to said reactor;
oxidizing said ammonia using said ammonia monooxygenase enzymes in said ammonia oxidizing bacteria to generate hydroxylamine and oxidizing said hydroxylamine using said hydroxylamine oxidoreductase enzymes to form nitrite;
partially oxidizing said methane in said compound using said ammonia monooxygenase enzymes in said ammonia oxidizing bacteria to generate methanol;
separating said methanol from other constituents in said reactor; and
collecting said methanol.

18. The method of claim 17, wherein oxidizing said ammonia is isolated from oxidizing said methane within said reactor.

19. The method of claim 17, wherein said bacteria include at least one of N. europaea, N. eutropha, Nitrosospira multiformis, N. oligotropha, Nitrobaster winogradskyi and Nitrospira spp.

20. The method of claim 17, further comprising continuously sparging said compound and said oxygen into said biomass in said reactor.
